(19) United States
(12) Patent Application Publication
(10) Pub. No.: US 2021/0043276 A1
(43) Pub. Date: Feb. 11, 2021

(54) METHODS OF IDENTIFICATION CONDITION-ASSOCIATED T CELL RECEPTOR OR B CELL RECEPTOR

(71) Applicant: MILABORATORY, LIMITED LIABILITY COMPANY, Moscow (RU)

(72) Inventor: Mikhail Valer'evich POGORELYY, Moscow (RU)

(21) Appl. No.: 16/979,617

(22) PCT Filed: Mar. 19, 2018
(86) PCT No.: PCT/RU2018/000168

§ 371 Date: Sep. 10, 2020

(51) Int. Cl. G16B 30/10

(52) U.S. Cl. CPC G16B 30/10

(57) ABSTRACT

Ability to efficiently identify T cell receptor (TCR) or B cell receptor (BCR) sequence variants associated with particular disease conditions is a prerequisite for development of wide diagnostics based on immune receptor repertoires analysis. It would also contribute to precise TCR- or BCR-based immunotherapies of infections and cancer, as well as to the treatment of autoimmune diseases and allergies by targeted elimination of disease-associated T cell or B cell clones. Provided herein are methods for using the data on immune receptor repertoires to identify condition-associated TCR or BCR sequence variants or groups of homologous variants in cohorts of patients with particular disease conditions, or undergoing particular therapy or vaccination.

### FIELD OF THE INVENTION

The invention relates to bioinformatics and medicine, namely methods for the identification of T cell or B cell receptor sequence variants or groups of homologous sequence variants associated with a disease conditions or conditions related to a therapy or vaccination.

### BACKGROUND OF THE INVENTION

The development of high-throughput sequencing techniques has made it possible to obtain and analyze repertoires of antigen-recognising receptors of T-lymphocytes (T-cell receptors, TCRs) and B-lymphocytes (B-cell receptors, BCRs, as used herein is equivalent to antibodies, or immunoglobulins).

A variety of TCR variants, as well as a variety of BCRs, is formed as a result of a relatively random “assembly” of gene segments—recombination events, during each of which one of the variants of the so-called V, D, and J segments available in the genome is selected to build a functional TCR beta chain or heavy antibody chain. TCR alpha chains and light antibody chains are generated similarly but without the use of D segments.

A relatively arbitrary combination of these gene segments is assembled into a new gene, with random deletion and addition of nucleotides at the junction of the gene segments. Thus, the junction of the segments is characterized by the greatest heterogeneity. Typically, the hypervariable regions, CDR3s, located at the junctions, subsequently plays a key role in antigen recognition.

An important feature of the recombination process is that different TCR and BCR sequence variants have different frequency of generation in the course of V(D)J-recombination [Murugan, A. et al. Proc Natl Acad Sci USA 16161-16166 (2012)]: some of the said sequence variants are generated frequently and may coincide in nucleotide and/or amino acid sequence for multiple T cell clones generated within single individual and in many individuals.

The connection between the sequence of TCR or BCR and its specificity, e.g. the particular recognized antigen, in most cases is unknown. Even if both the TCR or BCR sequence and the antigen are suggested, at the current state of the art it remains hardly possible to estimate would the affinity be sufficient to activate T or B cell.

At the same time, identifying TCR or BCR variants associated with certain diseases or other conditions is of great practical importance, since it potentially allows to 1) apply the analysis of BCR or TCR repertoires for diagnostics of infectious diseases, autoimmune diseases, allergic diseases, and cancer; 2) monitor consequences, efficiency, and long-term protection of vaccination; 3) identify TCR or BCR variants applicable for the specific therapy of infectious disease or cancer; 4) identify TCR or BCR variants associated with autoimmune or allergic disease as targets for therapeutic elimination.

It is known from the state of the art that oftenly the same or highly homologous immune receptor variants recognizing the same antigens are present in different donors [Miles, J. J. et al. Immunol. Cell Biol. 89, 375-387 (2011), Dash, P. et al. Nature 547, 89-93 (2017), Glanville, J. et al. Nature 547, 94-98 (2017).]. This makes it possible to search for associations of particular TCRs or BCRs or groups of TCRs or BCRs with homologous nucleotide or amino acid sequences with certain disease by comparing large cohorts of patients and healthy donors.

Based on this fact, the method was proposed in work [Emerson, R. O. et al. Nat. Genet. 49, 659-665 (2017)] for the search for TCR variants associated with positive serostatus in cytomegalovirus infection (CMV, cytomegalovirus). The method is the closest analog of the approach described in the present invention. In the proposed method, for each TCR variant having identical amino acid sequences in several donors, the uneven distribution of this clone between cohorts of patients and healthy donors is verified using Fisher's exact test. The 2×2 contingency table contains the number of patients and control donors with and without a given TCR amino acid sequence in the repertoire, respectively. Since this test is performed many times (for each TCR amino acid sequence found in several donors), the correction of the resulting p-values for multiple testing is introduced. This method has also been successfully applied to the search for the amino acid sequences of TCRs associated with the development of ankylosing spondylitis [Faham et al. Arthritis Rheumatol. 69, 774-784 (2017)]. Among the shortcomings of this method is the low power of Fisher test—cohorts of patients and healthy donors should be large enough that is not always achievable.

For many oncological or autoimmune diseases, high diversity of molecular targets recognized by immune receptors may lead to a high heterogeneity of associated TCR or BCR variants, thus expanding the required cohorts of patients and healthy control subjects.

Therefore, the important task remains to develop methods for more efficient identification of TCR or BCR variants or groups of variants associated with certain autoimmune, cancer, infectious, allergic or other disease conditions, vaccination, or therapy, that would be applicable for the limited cohorts of patients, and would not necessarily depend on the existence of healthy control cohorts.

### SUMMARY OF THE INVENTION

The present invention provides methods for using the data on immune receptor repertoires to identify condition-associated TCR or BCR variants or groups of homologous TCR or BCR variants in cohorts of individuals with particular disease or conditions related to a certain therapy or vaccination, that employs estimated frequency of generation of TCR or BCR sequences as a criteria of selection of the candidate condition-associated immune receptor variants or groups of variants.

For the purposes of the present invention, the length of the compared sequences may be the same as the lengths of the CDR3 regions of the TCR or BCR chains being analyzed, or may be extended to the portion of or to the whole sequences of V and/or J gene segments, or may represent a portion of CDR3 sequence.

The object of the present invention is to expand the means for detecting sequences of antigen-recognizing receptors associated with certain diseases or other conditions. The technical result of the present invention also consists in the emergence of a method for detecting sequences of antigen-recognizing receptors associated with certain diseases or conditions in limited cohorts of patients.

An important feature of the proposed method is that for the detection of TCR or BCR sequence variants associated with certain disease or condition, it is not necessary to obtain data from the sequencing of the immune receptors of the control cohort of healthy donors. The technical result is achieved due to a new algorithm for the analysis of antigen-recognition receptors, based on a comparison of the relative frequencies of nucleotide and/or amino acid sequences generation by immune system estimated in silico and their representation in experimental data.

In one aspect, the method for identifying TCR or BCR sequence variants associated with a disease condition, therapy-induced condition, or vaccination-induced condition in cDNA- or genomic DNA-based sequencing data obtained from samples from a cohort of individuals is provided comprising: identification of amino acid or nucleotide sequence variants shared between TCR or BCR repertoires of at least two individuals within a cohort of individuals with a given condition; in silico estimation of the relative frequency of genomic TCR or BCR rearrangements resulting in such sequence variants; identification of sequence variants for which the number of donors in which a sequence variant was identified is significantly higher than expected from the estimated relative frequency of genomic TCR or BCR rearrangements resulting in such sequence variant.

In another aspect, a method for identifying groups of homologous TCR or BCR sequence variants associated with a disease condition, therapy-induced condition, or vaccination-induced condition in cDNA- or genomic DNA-based sequencing data obtained from samples from a cohort of individuals is provided comprising: identification of groups of homologous amino acid or nucleotide sequence variants which members are shared between TCR or BCR repertoires of at least two individuals within a cohort of individuals with a given condition; in silico estimation of the relative frequency of genomic TCR or BCR rearrangements resulting in such sequence variants; identification of groups of homologous sequence variants for which the number of donors in which members of a group were identified is significantly higher than expected from the estimated relative frequency of genomic TCR or BCR rearrangements resulting in sequence variants that belong to a group.

In another aspect, a method for identifying TCR or BCR sequence variants associated with a disease condition, therapy-induced condition, or vaccination-induced condition in cDNA- or genomic DNA-based sequencing data obtained from samples from a cohort of individuals is provided comprising: identification of amino acid sequence variants shared between TCR or BCR repertoires of at least two individuals within a cohort of individuals with a given condition; in silico estimation of the relative frequency of genomic TCR or BCR rearrangements resulting in such amino acid sequence variants; identification of amino acid sequence variants for which the number of different nucleotide sequence variants identified in a cohort that encode an amino acid sequence variant is significantly higher than expected from the estimated relative frequency of genomic TCR or BCR rearrangements resulting in such amino acid sequence variant.

In another aspect, a method for identifying groups of homologous TCR or BCR sequence variants associated with a disease condition, therapy-induced condition, or vaccination-induced condition in cDNA- or genomic DNA-based sequencing data obtained from samples from a cohort of individuals is provided comprising: identification of groups of homologous amino acid sequence variants which members are shared between TCR or

BCR repertoires of at least two individuals within a cohort of individuals with a given condition; in silico estimation of the relative frequency of genomic TCR or BCR rearrangements resulting in amino acid sequence variants that belong to such groups; identification of groups of homologous amino acid sequence variants for which the number of different nucleotide sequence variants identified in a cohort that encode members of a group is significantly higher than expected from the estimated relative frequency of genomic TCR or BCR rearrangements resulting in amino acid sequence variants that belong to a group.

In another aspect, a method for identifying TCR or BCR sequence variants associated with a disease condition, therapy-induced condition, or vaccination-induced condition in cDNA- or genomic DNA-based sequencing data obtained from samples from a cohort of individuals is provided comprising: identification of amino acid or nucleotide sequence variants shared between TCR or BCR repertoires of at least two individuals within a cohort of individuals with a given condition; in silico estimation of the relative frequency of genomic TCR or BCR rearrangements resulting in such sequence variants; identification of sequence variants for which the number of identified homologous sequence variants observed in said individuals is significantly higher than expected from the estimated relative frequency of genomic TCR or BCR rearrangements resulting in such amino acid sequence variant.

In another aspect, a method for identifying groups of homologous TCR or BCR sequence variants associated with a disease condition, therapy-induced condition, or vaccination-induced condition in cDNA- or genomic DNA-based sequencing data obtained from samples from a cohort of individuals is provided comprising: identification of groups of homologous amino acid or nucleotide sequence variants which members are shared between TCR or BCR repertoires of at least two individuals within a cohort of individuals with a given condition; in silico estimation of the relative frequency of genomic TCR or BCR rearrangements resulting in such sequence variants; identification of sequence variants that belong to said groups of homologous amino acid or nucleotide sequence variants and for which the number of homologous subvariants is significantly higher than expected from the estimated relative frequency of genomic TCR or BCR rearrangements resulting in sequence variants that belong to a group.

In some embodiments, identification of TCR or BCR sequence variants or groups of homologous sequence variants for which the number of donors in which a sequence variant or members of said groups were identified, or the number of different nucleotide sequence variants identified in a cohort that encode an amino acid sequence variant or a group of homologous sequence variants, or a number of homologous subvariants identified in individuals of a cohort, is significantly higher than expected from the estimated relative frequency of genomic TCR or BCR rearrangements resulting in such sequence variant or a group of homologous variants, is performed using outlier detection technique. Such techniques are known from the state of the art, and include, but not limited to: Z-scores, DBSCAN [Schubert, E. et al. ACM Trans. Database Syst. 42, 1-21 (2017).], isolation forests [Liu, F. T. et al. ACM Trans. Knowl. Discov. Data 6, 1-39 (2012).], LOF [Breunig, M. M. et al. Proc. 2000 Acm Sigmod Int. Conf. Manag. Data 1-12 (2000).], SOD [H. Kriegel, et al. Proceeding 13th ACM SIGKDD Int. Conf. Knowl. Discov. Data Min 831-838 (2009).], ABOD [H. Kriegel, et al: Proceeding 14th ACM SIGKDD Int. Conf. Knowl. Discov. Data Min.—KDD 08, 444 (2008).], LOCI [S. Papadimitriou et al. Proc. 19th Int. Conf. Data Eng. 315-326 (2003)], INFLO [W. Jin et al. Lect. Notes Comput. Sci. 577-593 (2006).], RBRP [A. Ghoting et al. Data Min. Knowl. Discov. 16, 349-364 (2008)], ORCA [M. Schwabacher, S. D. Bay ACM Spec. Interes. Gr. Knowl. Discov. Data Data Min. (2003).], FDC [T. Johnson et al., Am. Assoc. Artifical Intell. 224-228 (1998).], ISODEPTH [I. Ruts, P. J. Rousseeuw, Comput. Stat. Data Anal. 23, 153-168 (1996).], ROF [H. Fan et al. Lect. Notes Comput. Sci. 557-566 (2006)], grid-based subspace outlier detection [C. C. Aggarwal et al.: Proc. 2001 ACM SIGMOD Int. Conf. Manag. Data, 37-46 (2001)], distance based method [E. M. Knorr et al. Complexity. (1997).], deviation based method [A. Arning et al. KDD-96 Proc. 164-169 (1996)] and others.

In some embodiments, identification of TCR or BCR sequence variants or groups of homologous sequence variants for which the number of individuals in which a sequence variant or members of said groups were identified, or the number of different nucleotide sequence variants identified in a cohort that encode an amino acid sequence variant or a group of homologous sequence variants, or a number of homologous subvariants identified in a individuals of a cohort, is significantly higher than expected from the estimated relative frequency of genomic TCR or BCR rearrangements resulting in such sequence variant or a group of homologous variants, is performed using other statistical methods.

In another aspect, a method for identifying TCR or BCR sequence variants contamination in a tissue sample or samples or in a sequencing data by comparing the diversity of simulated nucleotide sequence variants encoding the same amino acid variant to the diversity of such variants observed in individuals cohort is provided comprising: simulating generation of nucleotide sequence variants encoding amino acid sequence of interest; calculating frequency of each unique nucleotide variant generated; comparing frequency of each nucleotide variant of given amino acid sequence observed in data to frequency of this nucleotide variant in simulated data; identification of possible intersample contaminations as nucleotide sequence variants which frequency observed in data is significantly higher than in simulated data.

In silico estimation of the relative frequency of genomic rearrangements resulting in certain TCR or BCR sequence variants or groups of variants may be performed using various methods.

Existing mechanistic models of assembly of antigen-recognition receptors [Murugan, A. et al. Proc. Natl. Acad. Sci. 109, 16161-16166 (2012), Marcou, Q. et al. Nat. Commun. 9, 561 (2018)] can be used for estimation of relative frequency of rearrangements for the nucleotide TCR or BCR sequence variants.

These models do not allow to accurately estimate the relative frequency of rearrangements for amino acid TCR or BCR sequences. However, knowing the parameters of the assembly model and the sequence of genomic V-, D- ϰ J-segments, it is possible to generate nucleotide TCR or BCR sequence variants in silico.

Using the rules of the genetic code, the simulated nucleotide sequences obtained may be translated, resulting in simulated amino acid TCR or BCR sequence variants. The proportion of simulated amino acid sequences that coincide with the amino acid sequence from the experimental data is an estimate of the relative frequency of rearrangements resulting in the amino acid sequence from the data.

Thus, one way to theoretically predict the relative frequency of TCR or BCR sequence generation is to estimate the frequency of its appearance in an array of TCR or BCR sequences simulated using open source software [Marcou, Q. et al. Nat. Commun. 9, 561 (2018), Nazarov, V. I. et al. BMC Bioinformatics 16, (2015)].

It is also possible to estimate the relative frequency of amino acid TCR or BCR sequence generation without simulation nucleotide sequences or searching for a large array of healthy donor repertoires.

Since the methods exist to estimate the relative frequency of nucleotide TCR or BCR sequence generation [Murugan, A. et al. Proc. Natl. Acad. Sci. 109, 16161-16166 (2012), Marcou, Q. et al. Nat. Commun. 9, 561 (2018)], these methods may be applied to all possible nucleotide sequence variants encoding a given amino acid TCR or BCR variant. Possible nucleotide TCR or BCR variants encoding amino acid TCR or BCR variant can be obtained using the rules of the genetic code, enumerating all codons for each amino acid. Relative frequency of an amino acid TCR or BCR sequence generation can be further estimated as a sum of relative frequencies of corresponding nucleotide TCR or BCR sequences generation encoding such amino acid variant.

Another way of estimating the relative frequency of TCR or BCR sequence generation is to search for such sequences in large sets of data from the TCR or BCR repertoires of healthy donors, such as [Britanova et al., J Immunol., 196(12), 5005-5013. (2016)].

Since the main contribution to the variety of TCR and BCR variants is made by randomly added N nucleotides by Terminal deoxynucleotidyl transferase (TdT) enzyme at the junctions of V and J, and/or V and D, and/or D and J segments, the simplest estimation of relative frequency of a TCR or BCR variant generation may be based on the identification of the number of randomly added N nucleotides within the CDR3 region.

The methods of the present invention are applicable to cohorts of individuals comprising at least two individuals suffering from the same disease, or obtaining the same therapy or vaccination. Thus, a cohort of individuals for analysis includes two or more individuals, mainly three or more individuals, for example, 5 individuals or more, 10 individuals or more, more often 20 individuals or more.

In some embodiments, experimental data for implementing the method of the present invention are obtained by sequencing DNA or RNA samples isolated from a biological material obtained from individuals within one year after the initiation of a disease or event that elicits an immune response.

In some embodiments, the biological material is obtained within 1 to 45 days after the initiation of a disease or event that elicits a response from the immune system.

In some embodiments, the biological material is obtained after more than 1 year after the initiation of the disease or event that elicits the immune system response.

Further features and advantages of certain embodiments of the present invention will become more fully apparent in the following description of the embodiments and drawings thereof, and from the claims.

### BRIEF DESCRIPTION OF THE FIGURES

FIG. 1. Identification of previously validated CMV-specific clonotype. Number of in silico rearrangements for each clonotype in a TRBV7-6, TRBJ1-4 VJ combination is plotted against the number of patients with that clonotype. Known CMV specific clonotype CASSLAPGATNEKLFF is marked with the circle.

FIG. 2. Identification of ankylosing spondylitis-associated clonotype using proposed approach. Black points are clonotypes found exclusively in patients and absent in healthy control cohort. Grey points are clonotypes found both in patients and controls. Dashed line shows Z-test significance threshold.

### DETAILED DESCRIPTION OF THE PREFERRED EMBODIMENTS

Before the subject invention is described further, it is to be understood that the invention is not limited to the particular embodiments of the invention described below, as variations of the particular embodiments may be made and still fall within the scope of the appended claims. It is also to be understood that the terminology employed is for the purpose of describing particular embodiments, and is not intended to be limiting. Instead, the scope of the present invention will be established by the appended claims.

In this specification and the appended claims, the singular forms “a,” “an” and “the” include plural reference unless the context clearly dictates otherwise. Unless defined otherwise, all technical and scientific terms used herein have the same meaning as commonly understood to one of ordinary skill in the art to which this invention belongs.

Where a range of values is provided, it is understood that each intervening value, to the tenth of the unit of the lower limit unless the context clearly dictates otherwise, between the upper and lower limit of that range, and any other stated or intervening value in that stated range, is encompassed within the invention. The upper and lower limits of these smaller ranges may independently be included in the smaller ranges, and are also encompassed within the invention, subject to any specifically excluded limit in the stated range. Where the stated range includes one or both of the limits, ranges excluding either or both of those included limits are also included in the invention.

Unless defined otherwise, all technical and scientific terms used herein have the same meaning as commonly understood to one of ordinary skill in the art to which this invention belongs. Although any methods, devices and materials similar or equivalent to those described herein can be used in the practice or testing of the invention, the preferred methods, devices and materials are now described.

All publications and patents cited in this specification are herein incorporated by reference as if each individual publication or patent were specifically and individually indicated to be incorporated by reference. The citation of any publication is for its disclosure prior to the filing date and should not be construed as an admission that the present invention is not entitled to antedate such publication by virtue of prior invention.

In order to better understand the invention, certain terms are defined below.

It should be understood that the materials and methods proposed herein are not limited to particular compositions or process steps, as they may vary. It is pointed out that, as used in this specification and the appended claims, singular forms include the corresponding plural forms, unless the context clearly dictates otherwise.

The term “antigen-recognizing receptor” as used herein refers to TCRs and BCRs.

The term “immune receptor repertoires” as used herein refers to TCR and/or BCR repertoires.

The term “BCR” as used herein refers to B cell receptors, antibodies, or immunoglobulins.

As used herein, the term “CDR3” refers to complementarity determining regions—the hypervariable regions of the antigen-recognizing receptors located in the variable domains of the TCRs or BCRs chains.

Each of the chains of the antigen-recognizing receptor consists of three CDRs and four FR regions located from the amino- to carboxyl terminus in the following order: FR1, CDR1, FR2, CDR2, FR3, CDR3, FR4.

The terms “patient” and “individual” refer to a vertebrate, in particular to a representative of mammalian species, and include, but are not limited to, pets, sports animals, primates, including humans.

As used herein, the term “cohort of individuals” refers to a group of patients of the same species suffering from the same disease, or individuals undergoing the same type of therapy or vaccination. In preferred embodiments, the term refers to a group of people.

As used herein, the term “experimental data” refers to the sequencing data of biological samples obtained from individuals belonging to the analyzed cohort.

As used herein, the term “biological sample” refers to a sample of peripheral blood, a sample of cells or a tissue sample, for example, a biopsy or puncture material taken from a patient, and to cell cultures derived from patient cells.

In some embodiments, the cells of interest are cultured and differentiated in vitro before use.

The term “gene segments” is used to describe the segments of the genes that are involved in the generation of TCRs and BCRs.

The term “nucleotide sequences” refers to nucleic acid sequences, including DNA, such as genomic DNA or a cDNA molecule, or RNA. As used herein, the term “cDNA” refers to nucleic acids that have the sequence elements complement to the native mature mRNA species, where the sequence elements are exons and the 5 ′ and 3′ non-coding regions.

As used herein, the term “sample preparation” refers to all manipulations to which nucleic acids are subjected after purification and before sequencing.

The term “homology” is used to describe the interconnection of nucleotide or amino acid sequences with other nucleotide or amino acid sequences, which is determined by the degree of identity and/or similarity between the indicated sequences compared.

As used herein, the sequences are “highly homologous” if they differ by not more than three nucleotide or amino acid mismatches and/or indels, for example, they differ by three, or by two, or by one nucleotide or amino acid mismatch and/or indel along the entire length of the region chosen for comparison.

Two sequences that are identical to one another are also highly homologous.

For the purposes of the present invention, the length of the compared sequences may be the same as the lengths of the CDR3 regions of the TCR or BCR chains being analyzed, or may be extended to the portion of or to the whole sequences of V and/or J gene segments, or may represent a portion of CDR3.

Sequence comparison is performed by methods known to those skilled in the art. For example, the algorithm described in [Altschul et al. J. Mol. Biol., 215, pp. 403-10 (1990)] may be employed for sequence comparison.

As another example, to determine the level of identity and similarity between nucleotide or amino acid sequences, the Blast software package provided by National Center for Biotechnology Information (http:www.ncbi.nlm.nih.gov/blast).

Reference to the nucleotide sequence coding amino acid sequence means that this amino acid sequence is produced from the nucleotide sequence during translation of mRNA. As is obvious to any person skilled in the art, the term also includes degenerate nucleotide sequences encoding the same amino acid sequence.

“T cell receptor”, also referred to as “TCR”, is a heterodimeric protein complex located on the T lymphocyte surface. This receptor is present only on T lymphocytes. The main function of TCR is the specific recognition of processed antigens represented by molecules of the main histocompatibility complex (MHC, or HLA).

Human TCR consists of two subunits, α and β, or γ and δ chains, interconnected by a disulfide bond and presented on the T cell membrane. Each of the TCR chains has an N-terminal variable (V) domain, a junction domain (J) and a constant (C) domain coupled to a transmembrane domain that fixes the receptor in the plasma membrane of the T lymphocyte. The TCR usually and mostly interacts with the MHC antigen complex with six complementarity determining regions (CDRs): three alpha chain and three beta chain regions. In some cases, TCR alpha chain interaction with antigen dominates. More usually, TCR beta chain interaction with antigen dominates.

A small fraction of T-lymphocytes has receptors of the γ/δ type. They are similar in their design to α/β receptors, but differ in their primary structure and have a number of distinct functional features. They recognize antigens in a complex with “nonclassical” (not MHC) antigen-presenting molecules.

After the primary assembly of TCR, a large part (95-98%) of T lymphocytes die in the thymus as a result of positive and negative selection from the set of randomly generated TCR sequences. This strict selection is carried out only by those TCRs that, on the one hand, are principally capable of recognizing antigens in the context of MHC, and on the other, do not show strong interaction with MHC carrying self-peptides. Such selection is necessary in order to avoid autoimmune reactions—attacks of T cells on the host's own cells.

A significant portion of mature T-lymphocytes, in addition to functional TCRs, carries non-functional TCR chains on the second, homologous, chromosome, with a broken reading frame or with a stop codon. Such TCRs are not expressed on the surface of T cells, and therefore do not pass a positive and negative selection in the thymus. Thus, analysis of these sequences allows to obtain information about the process of recombination events during which the pre-selected repertoire of TCR variants is generated [Robins, H. S. et al. Sci Transl Med 47ra64 (2010), Murugan, A. et al. Proc Natl Acad Sci USA 16161-16166 (2012), Putintseva, E. V. et al. Front Immunol 463 (2013), Elhanati, Y. et al. Proc Natl Acad Sci USA 9875-9880 (2014), Zvyagin, I. V. et al. Proc Natl Acad Sci USA 5980-5985 (2014)]. This information includes the relative frequencies of the gene segments usage, from which the TCR is assembled, the number of randomly added and deleted nucleotides at the junctions of the segments, the length of the so called P-segments, that altogether determine the initial composition of TCR repertoire before selection for each individual and in the human population.

On the basis of data obtained from the non-functional TCR variants, probabilistic model of TCR generation was built [Elhanati, Y. et al. Proc Natl Acad Sci USA 9875-9880 (2014)]. Such model allows in silico generation of artificial TCR repertoires nearly identical to the real product of recombination machinery in humans.

B cell receptors (BCRs) represent a heterotetramer of two light and two heavy antibody chains, expressed on the B cell surface or secreted as a soluble antibody. The same basic principles of VJ (for the light chains) and VDJ (for the heavy chains) diversity generation are applicable for the BCR as discussed for TCRs above.

The methods of the present invention find application in the search for TCR and/or BCR sequence variants or groups of variants, which are associated with various diseases or conditions. For example, the methods finds application in the search for TCR and/or BCR variants or groups of variants that are associated with cancer, autoimmune, allergic diseases or are formed as a result of the immune response to acute or chronic infections or occur during vaccination or in response to a therapy.

For the purposes of the present invention, cDNA- or genomic DNA-based sequencing data obtained from samples from a cohort of individuals are used.

A sample of DNA or RNA can be isolated from a biological material, for example from cell cultures, tissues, swabs and scrapings, biopsy material, biological fluids (blood, urine, saliva) of animals, including humans.

In preferred embodiments, the biological material for isolating DNA or RNA is obtained from patients suffering from the same disease or individuals undergoing the same type of therapy or vaccination.

The methods of the present invention are applicable to cohorts of individuals comprising at least two individuals suffering from the same disease, or obtaining the same therapy or vaccination. Thus, a cohort of individuals for analysis includes two or more individuals, mainly three or more individuals, for example, 5 individuals or more, 10 individuals or more, more often 20 individuals or more. Increasing the number of individuals improves the accuracy of the analysis, so the group can be increased without restrictions.

In some embodiments, experimental data for implementing the method of the present invention are obtained by sequencing DNA or RNA samples isolated from a biological material obtained from individuals within one year after the initiation of a disease or event that elicits an immune response. In some embodiments, the biological material is obtained within 1 to 30 days after the initiation of a disease or event that elicits a response from the immune system. In some embodiments, the biological material is obtained for more than 1 year after the initiation of the disease or event that elicits the immune system response.

In preferred embodiments, patients included in a cohort belong to the same species and suffer from the same disease. The analysis of antigen-recognition receptors may be in demand for a disease selected from the group of: cancer, autoimmune diseases, infectious diseases, allergies, helminthic invasions, etc. In some embodiments, the individuals included in the cohort do not have the disease, but are vaccinated using the same vaccine or treated with the same therapy.

In some embodiments related to chronic diseases, the timing of the collection of the biomaterial from the patient is not directly related to the moment of onset of the disease, but is not less than one day from the moment of the onset or diagnosis of the disease.

In some embodiments involving the search for TCR or BCR sequence variants or groups of variants involved in an immune response against infections or associated with allergic reactions or with immune system reactions to vaccination, the biomaterial is collected within one year after the initiation of the disease or event that elicited the immune system response, or later.

In some embodiments, biomaterial is collected within 1 to 45 days after the initiation of a disease or event that elicited a response from the immune system.

Any methods known in the art can be used to isolate genomic DNA or RNA. For example, the methods described in Sambrook et al. [Molecular Cloning: A Laboratory Manual, 2nd edition. Cold Spring Harbor Laboratory Press, Cold Spring Harbor, N.Y., (1989)], or commercially available kits for isolation and purification of nucleic acids, such as DNeasy or RNeasy (Qiagen, Germany) or their analogs can be used.

RNA samples can be used to prepare cDNA and derived gene libraries by reverse transcription and PCR techniques known in the art, for example, the Gulber-Hoffman method [Gubler, U., Hoffman, B. J. Gene. 1983. V. 25. P. 263-269], methods using DNA ligase of phage T4 [Akowitz & Manuelidis Gene. 1989. V. 81. P. 295-306; Lukyanov et al. Biophys. Biochem. Res. Comm. 1997. V. 230, P. 285-288], other ligase, method of cDNA synthesis with template switch [Chenchik et al., (1998) Gene Cloning and Analysis by RT-PCR, ed. by P Siebert and J Larrick, BioTechniques Books, Natick, Mass., 305-319; Zhu et al. Biotechniques. 2001. 30, 892-897] and other methods.

Samples of cDNA or DNA are subjected to sample preparation procedures, which usually include the amplification by PCR and introduction of adapters for sequencing. During sample preparation, samples can be enriched with nucleic acid sequences encoding TCRs or BCRs.

A number of techniques are known from the state of the art, applicable for analysis of TCR and BCR repertoires using sequencing or high-throughput sequencing. These techniques allow to identify genomic sequences of dozens, hundreds, thousands, and millions of TCR and/or BCR variants in blood samples, cell subpopulations of interest, or tissue of interest of the studied human donors or model animals. These techniques may use genomic DNA (e.g. [Robins, H. S. et al. Blood 4099-4107 (2009)] or RNA as a starting material to obtain gene libraries of alpha chain or beta TCR chains, heavy or light antibody chains, paired alpha-beta chain TCR gene libraries, or paired light-heavy antibody chain libraries. TCR or BCR libraries preparation can be performed using multiplex PCR amplification with a set of gene-specific primers, or using 5′RACE, or using other methods, in a bulk volume or for the cells separated into individual microvolumes using multiwell plates or water-in-oil emulsion.

For example, TCR or BCR gene library can be generated using 5′RACE as described in a number of works, such as [Britanova et al., J Immunol., 196(12), 5005-5013. (2016)]. In brief, bulk RNA from a PBMC or sorted T or B cells is extracted using column-based method, such as RNeasy Micro Kit (Qiagen) or similar, or using Trizol-based protocols, or mRNA extracted using column-based method, such as Oligotex mRNA Mini Kit (Qiagen) or using other technique. cDNA synthesis is performed using primers specific to TCR or BCR constant region or using oligo-T containing primer, with reverse transcriptase capable of template switching, such as SmartScribe (Clontech) or Mint (Evrogen), and a template switch oligo. Further amplification of TCR or BCR gene libraries is carried out using primers specific to the template switch oligo and constant regions or J-regions of TCR or BCR genes.

Alternatively, PCR amplification from cDNA template can be performed with a multiplex set of primers specific to the TCR or BCR genes.

Alternatively, genomic DNA can be extracted using any available method and used as a template for PCR amplification with a multiplex set of primers specific to the TCR or BCR genes. One or several stages of PCR amplification can be used, with nested and/or step-out primers. Sequencing adapters may be introduced in the course of PCR or ligated afterwards. Some of the examples of obtaining experimental data are described in [Robins, H. S. et al. Blood 4099-4107 (2009), Freeman, J. D. et al. Genome Res 1817-1824 (2009), Mamedov, I. Z. et al. Front Immunol 456 (2013)].

Alternatively, sequencing data on transcriptome analysis, RNA-seq, or exome analysis, Exome-Seq, or any sequencing data that include at least fragments of TCR or BCR sequences can be employed to extract information about TCR or BCR repertoires to obtain experimental data for the purposes of the present invention using appropriate software solutions, e.g. MiXCR software RNA-Seq mode [Bolotin D. A. et al., Nat Biotechnol. 35(10), 908-911 (2017)], or other software with appropriate functional.

Sequencing of adapters and methods for their introduction into nucleic acids are also well known to those skilled in the art and provided by manufacturers of commercial kits for high-throughput sequencing.

Sequencing can be performed using sequencing platform of interest, such as a sequencing platform provided by Illumina® (e.g., the HiSeg™, MiSeg™ and/or Genome Analyzer™ sequencing systems); Ion Torrent™ (e.g., the Ion PGM™ and/or Ion Proton™ sequencing systems); Pacific Biosciences (e.g., the PACBIO RS II sequencing system); Life Technologies™ (e.g., a SOLiD sequencing system); Roche (e.g., the 454 GS FLX+ and/or GS Junior sequencing systems); or any other sequencing platform of interest.

The detection of sequences of CDR3 regions of antigen-recognizing receptors—TCRs or BCRs—in the sequencing data can be performed by any of the methods known in the art. For example, these regions can be identified by multiple comparison of sequencing data with known TCR and BCR databases.

In particular, multiple alignment of the test sequences with known sequences of antigen-recognition receptor sequences can be perfromed, the information of which is summarized in the IMGT database («The international ImMunoGeneTics information system», Lefranc M-P., Nucl Acids Res 2001; 29:207-209), available on the Internet at http://www.imgt.org. Multiple alignment can be performed using a software package IgBlast, of the National Center for Biotechnology Information (https://www.ncbi.nlm.nih.gov/igblast/).

TCR and/or BCR repertoires can be extracted from sequencing or high-throughput sequencing data using software of interest, such as MiXCR [Bolotin D A et al. Nature Methods, 2015, 12(5), 380-381], or any other software of interest.

The terms “sequencing data” or “experimental data” refer to sequencing data containing at least a small proportion of regions of TCRs or BCRs obtained by any method known in the art, and not to a method for their production.

The complete individual TCR or BCR repertoire is not available for observation, usually the researcher deals with a small sample of blood or tissue, which contains up to several million T cells or B cells. The probability of a clone falling into this small sample is determined by its frequency in full repertoire.

It is known that different TCR and BCR sequence variants have a different generation frequency as a result of the V(D)J-recombination process [Murugan, A. et al. Proc. Natl. Acad. Sci. 109, 16161-16166 (2012)]: some TCR and BCR sequence variants are generated very often and can coincide in a variety of different clones within the same repertoire.

Thus, there are two major mechanisms by which a specific TCR or BCR sequence can be detected with high probability in the repertoire of a particular sample:
1) This variant of the TCR or BCR sequence has high frequency of generation, and within this donor there are many different clones (including those that are naive, that have not yet met their antigen) with the same amino acid TCR or BCR sequence, and possibly with the same nucleotide sequence TCR or BCR, which are the result of independent recombination events, so one or more T cells or B cells with such TCR or BCR with a high probability fall into the sample.
2) This variant of the TCR or BCR sequence has relatively low frequency of generation, but the T cell or B cell clone carrying such receptor has met the cognate antigen and underwent clonal expansion, thus cells of the resulting expanded clone get into the sample with a high probability.

It is known that parameters of TCR and BCR generation during recombination are similar among people, and therefore the same TCR and BCR sequence can be generated in different individuals by the first mechanism. Identification of such frequently generated TCR or BCR sequence variants or groups of such homologous sequence variants in different patients most likely has no diagnostic value, since such sequences may be frequently generated in any individual, and their presence in multiple samples does not necessarily indicates antigen-specific clonal expansion.

However, if there is a variant or a group of homologous TCR or BCR variants in different donors with a sequence or sequences with low estimated frequency of generation, this indicates that these TCR or BCR variants were repeatedly captured due to the second mechanism, and their clonal expansion could result from response to the same antigen.

Therefore, determining the relative frequency of generation for a TCR or BCR sequence variant or a group of homologous TCR or BCR sequence variants identified in several patients or healthy individuals with a give condition is important to determine the possible association of such sequence variants with a disease or other condition.

TCR or BCR sequence variants with a relatively low frequency of generation, represented in a large number of donors, are of greatest interest from the point of view of association with certain disease condition.

After a theoretical estimate of the generation frequency for a TCR or BCR sequence variant or a group of sequence variants, it is necessary to identify the sequences in the data shared between individuals that have an unexpectedly low probability of generation.

To this end, different techniques for outlier detection known from the state of the art can be used, such as Z-scores, DBSCAN, isolation forests, LOF, SOD, ABOD, LOCI, INFLO, RBRP, ORCA, FDC, ISODEPTH, ROF, grid-based subspace outlier detection, distance based methods, deviation based methods, and others.

For all amino acid sequences shared among given number of donors logarithms of recombination frequency are normally distributed. This allows to use the Z-test for outlier detection: null hypothesis implies that log p N(mu, sigma) for all sequences shared among n donors, there p is the frequency of generation, mu and sigma are normal distribution parameters estimated as sample mean and sample standard deviation for observed sample of amino acid sequences shared among n donors. For each number of donors parameters of normal distribution are estimated independently, and then Z-test is used to calculate p-value. If number of clonotypes shared among n donors is less than 30, then we pool close numbers of donors, until sample size exceeds 30. This is necessary for reliable estimation of the sample mean and standard deviation used in Z-test. In example: if in a given repertoire dataset there are 20 TCR or BCR sequence variants shared among seven donors, and 10 sequence variants shared among 8 donors, then one should join these groups together and estimate sample mean and sample standard deviation for 30 sequence variants shared among 7 or 8 donors.

After calculating p-values with Z-test it is necessary to adjust them for multiple testing, i.e. using Bonferroni, Holm, or Benjamini-Hochberg approaches. After that clonotypes with adjusted p-values lower than chosen significance threshold (i.e. p<0.05) are claimed significant.

The method of the present invention allows to identify condition-associated TCR or BCR variants or groups of homologous TCR or BCR variants in cohorts of individuals with particular disease or conditions.

The disease or conditions of interest include chronic infections, acute infections, cancer, autoimmune diseases, allergic diseases; conditions caused by vaccination or immunotherapy and other conditions. All such diseases or conditions are associated with changes in immune system, in particular with appearance of disease- or condition-associated TCR- and\or BCR sequences.

The following examples are offered by way of illustration and not by way of limitation.

### EXAMPLES

### Example 1. Identification of TCR Variants Associated with Chronic Viral Infection

PBMC samples were obtained for a cohort of 289 CMV-positive donors. TCR beta repertoires were obtained by high-throughput sequencing of genomic DNA-based TCR libraries generated using multiplex PCR [Robins, H. et al. Blood 114, 4099-107 (2009)].

Expected frequency of generation was estimated for the TCR beta chain CDR3 amino-acid sequences that were shared between at least two CMV-positive donors. To this end, random generation and translation of massive numbers of TCR nucleotide sequences was performed using IGoR software [Marcou, Q. et al. Nat. Commun. 9, 561 (2018)] and mechanistic statistical model of V(D)J recombination [Murugan, A. et al. Proc. Natl. Acad. Sci. 109, 16161-16166 (2012)].

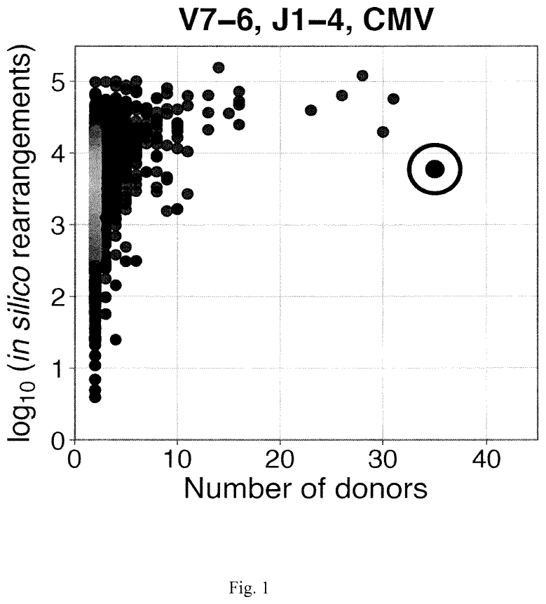

For each clonotype, the number of donors sharing that clonotype was plotted against its expected frequency of generation (FIG. 1). Previously validated CMV-specific TCR CASSLAPGATNEKLFF obtained from VDJdb database [Shugay, M. et al. Nucleic Acids Res. 46, 419-427 (2017).] had a 5.9 times lower expected frequency (p=0.03, Z-test) of generation than other sequences shared by more than 10 donors, confirming the reliability of the approach for identification of TCR variants associated with a chronic infection.

### Example 2. Identification of TCR Variants Associated with an Autoimmune Disease

Peripheral blood samples were obtained from 25 Ankylosing Spondylitis (AS) patients (24 HLA-B*27+) and 14 healthy donors (7 HLA-B*27+). Mononuclear cells were isolated by Ficoll density gradient centrifugation. Total RNA from PBMC was obtained using TRIzol reagent (Thermo Fisher Scientific). 5′-RACE TCR beta cDNA libraries were prepared according to the previously described general protocol [Mamedov, I. Z. et al. Front. Immunol. 4, 456 (2013)]. Libraries were sequenced with Illumina HiSeq 2000/2500 in 100+100 pair-end mode. TCR repertoires were extracted using MiTCR software [Bolotin, D. A. et al. Nat. Methods 10, 813-814 (2013)].

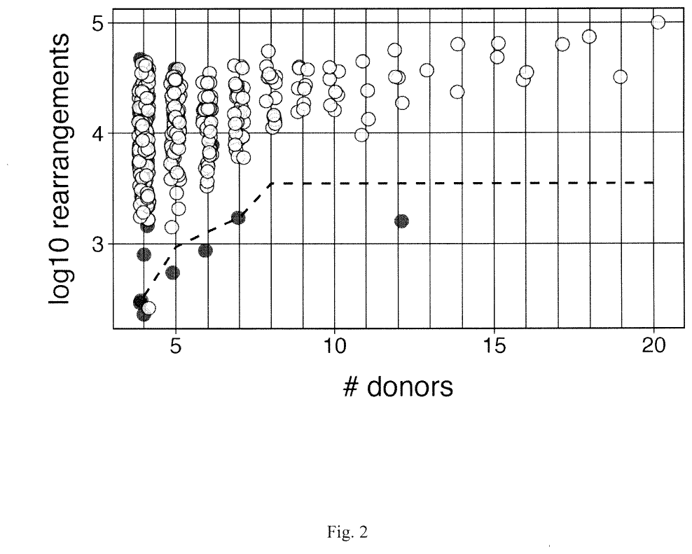

Sharing of the TRBV9_CASSVGLYSTDTQYF_TRBJ2-3 clonotype between 12 AS patients was observed. Generation probability of TCR CDR3 beta amino acid sequences encoded by TRBV9-TRBJ2-3 rearrangements for all clonotypes found in data was estimated as average number of corresponding nucleotide sequences generated in silico that encoded each TCR amino acid sequence. We generated 2×10\hat{ }9 TRBV9-TRBJ2-3 TCR rearrangements, out of which 1.2×10⁷ corresponded to 335 TRBV9-TRBJ2-3 CDR3 amino acid sequences shared between at least any 4 donors from the cohort: 25 AS patients, 7 HLA-B*27+, and 7 HLA-B*27− healthy donors. The number of donors positive for each particular clonotype was proportional to the number of in silico rearrangements that resulted in corresponding CDR3 amino acid sequence, thus demonstrating that clonotypes with higher recombination probability have higher sharing between donors (FIG. 2). Eight TCR beta clonotypes had higher detection frequency among donors than it could be expected by chance based on their recombination probability (p<0.001, Z-test dashed line on FIG. 2), suggesting their expansion driven by the common antigen. Seven of them had highly similar sequence and were found exclusively in AS patients. Of these clonotypes, the highest sharing between patients was observed for TRBV9_CASSVGLYSTDTQYF_TRBJ2-3, previously identified in synovial fluid of reactive arthritis patients [May, E. et al. Tissue Antigens 60, 299-308 (2002)] and also identified as AS-associated by comparing large cohorts of patients and healthy individuals [Faham, M. et al. Arthritis Rheumatol. 69, 774-784 (2017)].

### Example 3. Identification of Cancer-Associated Immunoglobulin Variants

Bulk RNA was extracted from tumor samples of 360 patients with skin cutaneous melanoma (SKCM). Bulk transcriptomic analysis (RNA-Seq) was performed, and immunoglobulin heavy chain (IGH) CDR3 repertoires were extracted using MiXCR software [Bolotin, D. A. et al. Nat. Methods 12, 380-381 (2015)] in RNA-Seq mode.

Clusters of homologous (having no more than 2 amino acid substitution) IGH CDR3 variants were built from the extracted repertoires.

Expected frequency of generation was estimated for IGH amino-acid CDR3 sequence clusters that were shared between at least three patients.

To this end, random generation and translation of massive numbers of IGH nucleotide sequences was performed using IGoR software [Marcou, Q. et al. Nat. Commun. 9, 561 (2018)] and mechanistic statistical model of V(D)J recombination [Elhanati, Y. et al. Philos. Trans. R. Soc. B Biol. Sci. 370, 20140243 (2015].

For each IGH amino-acid CDR3 sequence cluster, the number of donors sharing at least one CDR3 variant that composed the cluster was plotted against cumulative expected frequency of generation of amino acid CDR3 variants composing the cluster, and Z-score was calculated for sequences shared between each possible number of donors.

Those IGH amino-acid CDR3 sequence clusters that had significantly lower expected frequency of generation (p<0.05, Z-test, BH correction for multiple testing), than other clusters present in same number of donors were identified as associated with SKCM.

1. A method for identifying T cell receptor (TCR) or B cell receptor (BCR) sequences or groups of homologous TCR or BCR sequences associated with a given disease or condition in immune receptor repertoires data derived from cDNA- or genomic DNA-based sequencing data obtained from samples from a cohort of individuals, wherein the method comprises:
(A) identification of amino acid and/or nucleotide TCR or BCR sequences, satisfying at least one of the following criteria:
(I) amino acid TCR or BCR sequence is shared between immune receptor repertoires data of at least two individuals within a cohort of individuals with a given disease or condition;
(II) nucleotide TCR or BCR sequence is shared between immune receptor repertoires data of at least two individuals within a cohort of individuals with a given disease or condition;
(III) amino acid TCR or BCR sequence belongs to a group of homologous sequences, which members are represented in immune receptor repertoires data of at least two individuals with a given disease or condition, where homologous are considered those amino acid sequences that differ by up to three substitutions and/or indels;
(IV) nucleotide TCR or BCR sequence belongs to a group of homologous sequences, which members are represented in immune receptor repertoires data of at least two individuals with a given disease or condition, where homologous are considered those nucleotide sequences that differ by up to three substitutions and/or indels;
(B) estimation of the expected frequency of genomic TCR or BCR rearrangements resulting in sequences selected at stage (A);
(C) identification of TCR or BCR sequences or groups of homologous TCR or BCR sequences associated with a given disease or condition, as satisfying at least one of the following criteria:
(I) The number of individuals with a given disease or condition in which a TCR or BCR sequence or members of a group of homologous TCR or BCR sequences were identified at stage (A) is significantly higher than expected from the estimated frequency of genomic TCR or BCR rearrangements resulting in such sequence or sequences obtained at stage (B), where statistical significance is determined using outlier detection technique;
(II) The number of different nucleotide sequence variants encoding a TCR or BCR amino acid sequence or members of a group of homologous amino acid TCR or BCR sequences identified at stage (A) in individuals with a given disease or condition is significantly higher than expected from the estimated frequency of genomic TCR or BCR rearrangements resulting in such amino acid sequences obtained at stage (B), where statistical significance is determined using outlier detection technique;
(III) The number of observed homologous sequence subvariants of a TCR or BCR sequence identified in individuals with a given disease or condition at stage (A) is significantly higher than expected from the estimated frequency of genomic TCR or BCR rearrangements resulting in such sequence or sequences obtained at stage (B), where statistical significance is determined using outlier detection technique, and homologous are considered those sequence subvariants that differ by up to three substitutions and/or indels.

2. The method of claim 1, wherein said method further comprising the step of prioritizing TCR or BCR sequences or groups of homologous TCR or BCR sequences associated with a given disease or condition depending on the extent of clonal expansion within peripheral blood or tissue or cell subset of interest or cell culture within 1-45 days after condition or disease initiation.

3. The method of claim 1, wherein said method further comprising the step of prioritizing TCR or BCR sequences or groups of homologous TCR or BCR sequences associated with a given disease or condition depending on the extent of clonal contraction within peripheral blood or tissue or cell subset of interest in the period 10-300 days after condition or disease initiation.

4. The method of claim 1, wherein immune receptor repertoires data are initially split by V- and/or J-segments combinations.

5. The method of claim 1, wherein outlier detection technique is selected from the following:
Z-scores,
density-based spatial clustering of applications with noise (DBSCAN),
isolation forests,
isolation-based anomaly detection.
mining Distance-Based Outliers (ORCA)
local outlier factor (LOF),
ordering points to identify the clustering structure (OPTICS)
subspace outlier degree (SOD),
angle-based outlier degree (ABOD),
local outlier correlation integral (LOCI),
influenced Outlierness (INFLO),
algorithm for fast mining of distance-based outliers (RBRP),
fast computation of 2-dimensional depth contours (FDC),
depth contours of bivariate point clouds (ISODEPTH),
resolution-Based Outlier Factor (ROF),
grid-based subspace outlier detection.

6. The method of claim 1, wherein the disease is a chronic infection.

7. The method of claim 1, wherein the disease is an acute infection.

8. The method of claim 1, wherein the disease is a cancer.

9. The method of claim 1, wherein the disease is an autoimmune disease.

10. The method of claim 1, wherein the disease is an allergic disease.

11. The method of claim 1, wherein the condition is a vaccination.

12. The method of claim 1, wherein the condition is an immunotherapy.

13. A method of contamination control by comparing the diversity of simulated nucleotide TCR or BCR sequence variants encoding the same amino acid sequence variant to the diversity of such variants observed in a cohort of individuals, the method comprising the steps of:
A. Simulating generation of nucleotide sequence variants encoding amino acid sequence of interest;
B. Calculating a frequency of each unique nucleotide variant generated on step A) in simulated dataset;
C. Comparing a frequency of each nucleotide variant of given amino acid sequence observed in a cohort of individuals data to a frequency of this nucleotide variant in simulated data for difference in proportions; and
D. Considering the clonotype to be the result of intersample contamination if frequency of nucleotide variant in data is higher, than in simulated data.
